(19) United States
(12) Patent Application Publication
(10) Pub. No.: US 2010/0227921 A1
(43) Pub. Date: Sep. 9, 2010

(54) AMINO ACID AND PEPTIDE CARBAMATE PRODRUGS OF TAPENTADOL AND USES THEREOF

(75) Inventors: Richard Franklin, Hampshire (GB); Bernard T. Golding, Newcastle upon Tyne (GB); Robert G. Tyson, Durham (GB)

Correspondence Address: MCDERMOTT WILL & EMERY LLP, 600 13TH STREET, N.W., WASHINGTON, DC, 20005-3096, US

(73) Assignee: SHIRE LLC, Florence, KY (US)

(21) Appl. No.: 12/714,828

(22) Filed: Mar. 1, 2010

Related U.S. Application Data
(60) Provisional application No. 61/209,169, filed on Mar. 3, 2009.

(51) Int. Cl. A61K 31/27; C07C 271/54; A61P 29/00

(52) U.S. Cl. 514/490; 560/163

(57) ABSTRACT

Prodrugs of tapentadol with amino acids or short peptides, pharmaceutical compositions containing such prodrugs and a method for providing pain relief with the tapentadol prodrugs are provided herein. Prodrugs having side chains of valine, leucine, isoleucine and glycine amino acids and mono-, di- and tripeptides thereof are preferred. Additionally, methods for avoiding or minimizing the adverse gastrointestinal side effects associated with tapentadol administration, as well as increasing the oral bioavailability of tapentadol are provided herein.

### CROSS REFERENCE TO PRIOR U.S. APPLICATION

This application claims benefit under 35 U.S.C. §119(e) to U.S. Provisional Application No. 61/209,169, filed Mar. 3, 2009, which is hereby incorporated by reference in its entirety.

### FIELD OF THE INVENTION

The present invention relates to amino acid and peptide prodrugs of tapentadol to improve its oral bioavailability and pharmacokinetics, thereby enabling a reduction in inter-subject and intra-subject variability in plasma drug levels and analgesic response. Additionally, the invention achieves reduction in adverse gastrointestinal (GI) side-effects typically associated with tapentadol administration. These combined advantages should improve patient compliance and hence drug effectiveness of tapentadol in relieving pain.

### BACKGROUND OF THE INVENTION

Appropriate treatment of pain continues to represent a major challenge for both patients and healthcare professionals. Optimal pharmacologic management of pain requires selection of the appropriate analgesic drug that achieves rapid efficacy with minimal side effects. Full agonist opioid analgesics offer perhaps the most important option in the treatment of nociceptive pain, and remain the gold standard of treatment. However, misuse and abuse of opioids is a widespread problem and may deter physicians from prescribing these drugs.

Tapentadol is a mixed mu (μ) opioid agonist/norepinephrine re-uptake inhibitor of demonstrated clinical utility in the treatment of moderate to moderately severe pain (Tzschentke et al. (2007). J. of Pharmacol and Exp Ther. 323, 265-276 and Stegmann et al. (2008). Current Med. Res. Opin. 24, 3185-3196). Its structure is shown below.

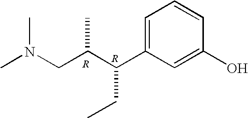

### Tapentadol

### (−)-(1R,2R)-3-(3-dimethyl-amino-1-ethyl-2-methyl-propyl)-phenol

In the U.S., an immediate release oral tablet of tapentadol hydrochloride is FDA approved for the treatment of moderate to severe acute pain. It is available in 50, 75 and 100 mg dosage forms. Adverse side effects associated with tapentadol include nausea, vomiting, constipation and dizziness.

Tapentadol is a high clearance drug (C1=1468±122 mL/min) and consequently exhibits low oral bioavailability (Terlinden et al. (2007). Eur. J. Drug Met and Pharmacokinetics 32, 163-169). This high clearance results in wide inter-patient variability in plasma drug concentrations (relative standard deviation in Cmax is 46%) and therefore, a uniform patient response may be lacking Tapentadol also has a relatively short plasma half-life in humans of 4.5 hours.

In addition to the issue of high clearance and inter-subject variability, tapentadol exhibits typical opioid GI side-effects of nausea/vomiting and constipation. Although less than for example, oxycodone, tapentadol (75 mg immediate release dosage) still induces nausea/vomiting in some 30-35% of patients. Constipation was evident in some 11% of treated patients (Afilalo et al., Poster No. 222 at Annual American Pain Society Meeting, May 2008). Such side-effects can lead to poor patient compliance and may even be dose limiting, denying the patient the full benefit of the drug.

There therefore remains a need in the treatment of moderate to moderately severe pain for a tapentadol product which retains all the inherent pharmacological advantages of the drug molecule but overcomes its principal limitations of poor pharmacokinetics and adverse GI side-effects.

### SUMMARY OF THE INVENTION

In one embodiment, the present invention is directed to a compound of Formula I, specifically a phenolic carbamate linked amino acid/peptide prodrug of tapentadol:

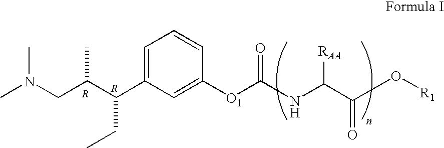

or a pharmaceutically acceptable salt thereof,

wherein,

O₁ is the phenolic oxygen atom present in the unbound tapentadol;

R₁ is selected from hydrogen, an unsubstituted alkyl group, or a substituted alkyl group;

n is an integer selected from 1 to 9; and

R_{AA} is a natural or non-natural amino acid side chain, and each occurrence of R_{AA} can be the same or different.

In one embodiment, n is 1, 2 or 3 while R₁ is H.

In another embodiment, n is 1, 2, 3, 4 or 5. In a preferred embodiment, the prodrug moiety of a tapentadol compound of the present invention has one or two amino acids (i.e., n is 1 or 2). In one embodiment, n is 3.

In a preferred embodiment, n is 1, 2 or 3 while R₁ is H. In another embodiment, n is 1. In yet another embodiment, n is 2. In yet another embodiment, n is 1 or 2 and each occurrence of R_{AA} is independently a natural amino acid side chain.

In another embodiment, a pharmaceutical composition comprising at least one tapentadol prodrug is provided. Specifically, the pharmaceutical composition comprises an effective amount of one or more of the tapentadol prodrugs of Formula I (or pharmaceutically acceptable salts thereof) and one or more pharmaceutically acceptable excipients.

Another embodiment is a method of treating pain in a subject in need thereof with tapentadol. The method comprises orally administering an effective amount of a tapentadol prodrug of the present invention to the subject. For example, the pain may be neuropathic pain or nociceptive pain. Other specific types of pain which can be treated with the tapentadol prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis, osteoarthritis or rheumatoid arthritis, and pain associated with cancer or its treatment.

In another embodiment of the invention, a method for increasing the oral bioavailability of tapentadol in a subject in need thereof is provided. The method comprises administering to a subject in need thereof an effective amount of a tapentadol prodrug of the present invention, or a composition thereof, wherein the oral bioavailability of tapentadol provided by the prodrug is at least 10% greater than the oral bioavailability of tapentadol when a molar equivalent of tapentadol is administered alone. An effective amount of the tapentadol prodrug is an amount sufficient to provide an analgesic response.

In one embodiment, a method for reducing inter- and/or intra-subject variability of tapentadol serum levels is provided. The method comprises administering to a subject, or group of subjects, in need thereof, an effective amount of a prodrug of the present invention, or a composition thereof. An effective amount of the tapentadol prodrug typically is an amount sufficient to provide an analgesic response.

In one embodiment, the present invention is directed to a method for minimizing the gastrointestinal (GI) side effects normally associated with oral administration of tapentadol. The method comprises orally administering a tapentadol prodrug or pharmaceutically acceptable salt of the present invention, and wherein upon oral administration, the prodrug or pharmaceutically acceptable salt minimizes, if not completely avoids, the gastrointestinal side effects usually seen after oral administration of the unbound tapentadol. The amount of tapentadol is preferably a therapeutically effective amount (e.g., an analgesic effective amount).

The present invention relates to natural and/or non-natural amino acids and short-chain peptides conjugated to tapentadol. Without wishing to be bound to any particular theory, the prodrugs presented herein can temporarily protect tapentadol from elimination during, for example, first pass metabolism. In addition, the prodrugs provided herein deliver a pharmacologically effective amount of the drug for the reduction or elimination of pain. The prodrugs of the present invention provide a viable means for increasing the bioavailability of tapentadol which, when administered alone, has a low bioavailability. Such use of prodrugs of tapentadol reduces intra- and inter-subject variability in plasma concentration and so provides consistent analgesic efficacy. Additionally, the presence of quantities of unhydrolyzed prodrug in plasma provides a reservoir for continued generation of the active drug (i.e., tapentadol). Continued generation of tapentadol maintains plasma drug levels, thereby reducing the frequency of drug dosage. Furthermore reduction of GI side-effects would be expected as the result of avoidance of direct interaction between active drug and opioid receptors in the gut. These benefits would be expected to improve patient compliance.

These and other embodiments are disclosed or are apparent from and encompassed by, the following Detailed Description.

### BRIEF DESCRIPTION OF THE DRAWINGS

FIG. 1 shows the tapentadol plasma concentration vs. time profile in dogs after oral administration of either tapentadol itself (1 mg tapentadol base/kg), or tapentadol valine carbamate (0.8 mg tapentadol free base equivalents/kg);

FIG. 2 is a graph of the log concentration of tapentadol or tapentadol valine carbamate (expressed as the free base of tapentadol) addition to isolated guinea pig ileum preparations, and the effects on electrical field stimulation response.

FIG. 3 is a graph of tapentadol mean plasma concentration (ng/mL) vs. time profile in male rats after oral administration of tapentadol hydrochloride (10 mg tapentadol base/kg).

FIG. 4 is a graph of tapentadol mean plasma concentration (ng/mL) vs. time profile in male rats after oral administration of tapentadol valine carbamate (10 mg tapentadol base/kg).

FIG. 5 is a graph of tapentadol valine carbamate mean plasma concentration (ng/mL) vs. time profile in male rats after oral administration of tapentadol valine carbamate (10 mg tapentadol base/kg).

FIG. 6 is a graph of tapentadol valine carbamate mean plasma concentration (ng/mL) vs. time profile in male monkeys after oral administration of tapentadol valine carbamate (1 mg tapentadol base/kg).

### DETAILED DESCRIPTION OF THE INVENTION

### Definitions

As used herein:

The term “peptide” refers to an amino acid chain consisting of 2 to 9 amino acids (bound via peptide bonds), unless otherwise specified. In preferred embodiments, the peptide used in the present invention is 2 or 3 amino acids in length. The present invention also concerns branched peptides, where an amino acid can be bound to another amino acid's side chain.

An amino acid is a compound represented by NH₂—CH(R_{AA})—COOH, wherein R_{AA} is an amino acid side chain (e.g., when R_{AA}═H, the amino acid is glycine). The term amino acid side chain, as used herein, is the substituent on the alpha-carbon of an amino acid.

The amino acids contemplated for use in the prodrugs of the present invention include both natural and non-natural amino acids. In one preferred embodiment, the amino acids are natural amino acids. The side chains R_{AA} can be in either the (R) or the (S) configuration. Both L- and D- amino acids are within the scope of the present invention.

A “natural amino acid” is one of the twenty two amino acids used for protein biosynthesis as well as other amino acids which can be incorporated into proteins during translation (including pyrrolysine and selenocysteine). A natural amino acid generally has the formula

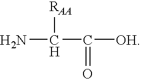

R_{AA}, in the case of a natural amino acid, is referred to as the natural amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine. The natural amino acids are also referred to as “proteinogenic amino acids.”

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

A “non-natural amino acid” is an organic compound which is an amino acid, but is not among those encoded by the standard genetic code, or incorporated into proteins during translation. Non-natural amino acids, thus, include amino acids or analogs of amino acids other than the 22 natural amino acids and include, but are not limited to, the D-isostereomers of amino acids. Examples of non-natural amino acids include, but are not limited to: citrulline, homocitrulline, hydroxyproline, homoarginine, homoserine, homotyrosine, homoproline, ornithine, 4-amino-phenylalanine, sarcosine, biphenylalanine, homophenylalanine, 4-amino-phenylalanine, 4-nitro-phenylalanine, 4-fluoro-phenylalanine, 2,3,4,5,6-pentafluoro-phenylalanine, norleucine, cyclohexylalanine, α-aminoisobutyric acid, N-methyl-alanine, N-methyl-glycine, N-methyl-glutamic acid, tert-butylglycine, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine, dipropylglycine and derivatives thereof wherein the amine nitrogen has been mono- or di-alkylated. Non-natural amino acids are also referred to as “non-proteinogenic amino acids.”

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (O) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl refers to a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulfur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “carbonyl” refers to a group —C(═O).

The term “carboxyl” refers to a group —CO₂H and consists of a carbonyl and a hydroxyl group (More specifically, C(═O)OH).

The terms “carbamate group,” and “carbamate,” concerns the group

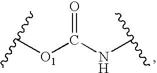

wherein the —O₁— is the phenolic oxygen in the unbound tapentadol molecule. Prodrug moieties described herein may be referred to based on their amino acid or peptide and the carbamate linkage. The amino acid or peptide in such a reference should be assumed to be covalently bound via an amino terminus on the amino acid or peptide to the carbonyl linker and the opioid analgesic, unless otherwise specified.

For example, val carbamate (valine carbamate) would have the formula

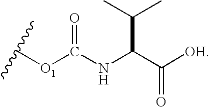

For a peptide, such as tyrosine-valine carbamate, it should be assumed, unless otherwise specified, that the leftmost amino acid in the peptide is at the amino terminus of the peptide, and is bound via the carbonyl linker to the opioid analgesic to form the carbamate prodrug.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E.W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be analgesia and/or an amelioration of one or more gastrointestinal side effect symptoms that are present when tapentadol in the prodrug is administered in its active form (i.e., when tapentadol is administered alone). It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to the tapentadol portion of the prodrug, as described herein.

Tapentadol is a chiral molecule containing two stereogenic centers and can therefore exist as four enantiomeric forms namely (R,R)—, (S,S)—, (S,R)—, and (R,S)— isomers of which the (R,R)— isomer is currently the clinically used form. Reference to tapentadol for the purposes of this invention, unless otherwise indicated, encompasses each enantiomer and mixtures thereof including a racemic mixture (racemate) of the enantiomers. The amino acid and peptide derivatives of tapentadol disclosed in the present invention can be either single isomers or mixtures of such isomers.

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1).

The term “bioavailability,” as used herein, generally refers to the rate and/or extent to which tapentadol is absorbed from a tapentadol product and becomes systemically available, and hence available at the site of action. See Code of Federal Regulations, Title 21, Part 320.1 (2003 ed.). For tapentadol oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of a drug given orally that reaches the systemic circulation after a single administration to a subject. A preferred method for determining the oral bioavailability is by dividing the AUC of the drug given orally by the AUC of the same dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “increase in oral bioavailability” refers to the increase in the bioavailability of tapentadol when orally administered as a prodrug of the present invention, as compared to the bioavailability when unbound tapentadol is orally administered. The increase in oral bioavailability can be from 5% to 5000%, from 50% to 5000%, from 500% to 5000%, or from 1000% to 5000%. At least 10%, at least 20%, at least 30%, at least 40%, at least 50%, at least 60%, at least 70% and at 80% increase in oral bioavailability is also encompassed by the term. Additionally, an increase in oral bioavailability by 2 (200%) to 10 times (i.e., a 1000% increase in bioavailability) is also encompassed by the term.

The term “low oral bioavailability,” refers to an oral bioavailability wherein the fraction of a dose of the parent drug given orally that is absorbed into the plasma unchanged after a single administration to a subject is 25% or less (e.g., 15% or less, or 10% or less). Without wishing to be bound by any particular theory, it is believed that the low oral bioavailability of tapentadol is the result of the conjugation of the phenolic oxygen to glucuronic acid during first pass metabolism. However, other mechanisms may be responsible for the decrease in oral bioavailability and are contemplated by the present invention.

### Compounds of the Invention

The prodrugs of the present invention are novel amino acid and peptide prodrugs of tapentadol linked via a carbamate group. Preferably, these prodrugs comprise tapentadol attached to a single amino acid or short peptide, via its phenolic oxygen. This modification to tapentadol improves the otherwise poor oral bioavailability of the drug.

In one embodiment of the present invention, the prodrugs are novel amino acid and peptide prodrugs of tapentadol, and are represented by Formula I.

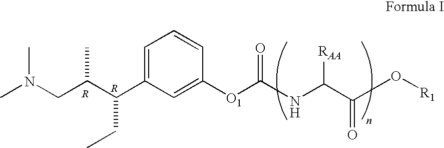

or a pharmaceutically acceptable salt thereof,

wherein,

O₁ is the phenolic oxygen atom present in the unbound tapentadol;

R₁ is selected from hydrogen, an unsubstituted alkyl group, or a substituted alkyl group;

n is an integer selected from 1 to 9; and

R_{AA} is a natural or non-natural amino acid side chain, and each occurrence of R_{AA} can be the same or different.

In one embodiment, n is 1, 2 or 3 while R₁ is H.

In one embodiment, n is 1, 2, 3, 4 or 5. In a preferred embodiment, the prodrug moiety of a tapentadol compound of the present invention has one or two amino acids (i.e., n is 1 or 2). In one embodiment, n is 3.

In a preferred embodiment, n is 1, 2 or 3 while R₁ is H. In another embodiment, n is 1. In yet another embodiment, n is 2. In yet another embodiment, n is 1 or 2 and each occurrence of R_{AA} is independently a natural amino acid side chain.

In one embodiment, n is 2 and at least one R_{AA} is bound to an additional amino acid, thereby forming a branched peptide.

In one embodiment, the compound of Formula I provides at least 10% greater oral bioavailability of tapentadol when compared to unbound tapentadol.

Preferred embodiments of the tapentadol prodrugs of Formula I are prodrugs wherein the side chain comprises a non-polar or an aliphatic amino acid. One such prodrug, tapentadol valine carbamate, is represented below:

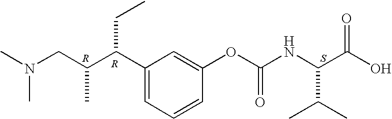

2-[3-(3-Dimethylamino-1-ethyl-2-methyl-propyl)-phenoxycarbonylamino]-3-methyl-butyric acid (tapentadol valine carbamate)

The preferred amino acids for use in the present invention are in the L configuration. However, the present invention also contemplates prodrugs of Formula I comprised of amino acids in the D configuration, or mixtures of amino acids in the D and L configurations.

Preferred embodiments of the carbamate linked prodrugs of tapentadol include, but are not limited to, tapentadol-(S)-isoleucine carbamate, tapentadol-(S)-leucine carbamate, tapentadol-(S)-aspartic acid carbamate, tapentadol-(S)-methionine carbamate, tapentadol-(S)-histidine carbamate, tapentadol-(S)-tyrosine carbamate, tapentadol-(S)-serine carbamate and pharmaceutically acceptable salts thereof.

Dipeptide prodrugs of tapentadol include tapentadol-(S)-valine-(S)-valine carbamate, tapentadol-(S)-isoleucine-(S)-isoleucine carbamate, tapentadol-(S)-leucine-(S)-leucine carbamate and pharmaceutically acceptable salts thereof.

In one embodiment, a prodrug of Formula I can include prodrug moieties comprising one or more of the following amino acids-valine, leucine, isoleucine, alanine and glycine. Further embodiments can include prodrug permutations drawn from these and other nonpolar aliphatic amino acids, with aromatic amino acids tryptophan and tyrosine.

Peptides comprising any of the natural amino acids are contemplated as prodrug moieties for use with the present invention. The 22 natural amino acids used for protein biosynthesis, as well as their abbreviations, are given in Table 1 below.

TABLE 1
Natural Amino Acids and Their Abbreviations

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |
| Selenocysteine | SEC | U |
| Pyrrolysine | PYL | O |

In one embodiment, a non-natural amino acid may be used as a prodrug moiety of the present invention (or portion thereof), either as either a single amino acid, included in a dipeptide or another short peptide. In the peptide embodiments, the peptide can contain only non-natural amino acids, or a combination of natural and non-natural amino acids.

### Advantages of the Tapentadol Prodrugs of the Present Invention

The use of the tapentadol prodrugs of the present invention preferably increases the oral bioavailability of the drug by 0.5 to 10 times (i.e., a 50 to 1000% increase in oral bioavailability), but lower increases in bioavailability are also within the scope of the invention. Without wishing to be bound to any particular theory, it is believed that the amino acid or peptide portion of the tapentadol prodrugs selectively exploit the inherent di- and tripeptide transporter Pept1 within the digestive tract to effect absorption. Once absorbed, these prodrugs provide sufficient temporary protection against the hepatic conjugation of tapentadol's phenolic functionality with glucuronic acid to ensure that a significantly larger amount of the drug reaches systemic circulation. It is believed that tapentadol is released from the amino acid or peptide prodrug by hepatic and extrahepatic hydrolases that are, in part, present in blood and or plasma.

Additionally, the use of the prodrugs of the present invention can provide greater consistency in analgesic response as the result of higher, more consistent, oral bioavailability. As a result of this more reproducible oral bioavailability, the prodrugs of the present invention offer a significant reduction of inter- and intra-subject variability of tapentadol plasma and CNS concentrations and, hence, significantly less fluctuation in pain relief for a single patient, or among a patient population. Thus, patient compliance is likely to be further improved as the result of this greater predictability of analgesic response.

Adverse GI side-effects of nausea/vomiting and constipation associated with opioids have historically represented serious limitations to their use. Tapentadol, while being associated with somewhat fewer adverse effects than other opioids, nevertheless, still induces significant emesis and constipation. Opioid-induced constipation induced is not only a distressing condition, but is often severe enough to be dose limiting, and therefore can interfere with adequate pain control (Shiova et al. (2007). Palliative and Supportive Care 5, 161-166). A significant number of patients receiving long term opioid therapy would rather endure their pain than the severe incapacitating constipation (Vanegas (1998). Cancer Nursing 21, 289-297).

It is possible that part or all of tapentadol's constipating and adverse GI side effects are due to its direct actions on the opioid receptors in the gut. Several recent studies have shown that regionally confined (i.e., within the gut lumen) narcotic antagonists such as alvimopan and naloxone, effect a profound reduction in the constipating effects of orally administered opioids.

For example, one recent study showed that alvimopan reversed codeine's inhibitory effects on gut motility (Goenne et al. (2005). Clin Gastroenterology and Hepatology 3, 784-791). Additionally, co-administration of alvimopan, methylnaltrexone and naloxone with opioid analgesics such as oxycodone has shown a reduction in effects on gut transit, without adversely affecting systemically mediated analgesia (Linn and Steinbrook (2007). Tech in Reg. Anaes. and Pain Management 11, 27-32). Thus, oral administration of a transiently inactivated tapentadol may similarly avoid such problems of locally mediated constipation, without the need for co-administration of a peripheral μ-opioid antagonist, as the prodrug would preclude access of active drug species to the g-opioid receptors within the gut wall.

### USES OF THE INVENTION

In one embodiment, a method is provided for treating pain with tapentadol in a subject in need thereof. The method comprises orally administering an effective amount of a tapentadol prodrug of the present invention to the subject. For example, the pain may be neuropathic pain or nociceptive pain. Specific types of pain which can be treated with the tapentadol prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis, osteoarthritis or rheumatoid arthritis, and pain associated with cancer or its treatment. The prodrug can be any tapentadol prodrug encompassed by Formula I. The amount of tapentadol is preferably a therapeutically effective amount (e.g., an analgesic effective amount).

In one embodiment, the present invention is directed to a method for minimizing the gastrointestinal side effects normally associated with oral administration of tapentadol. The method comprises orally administering a tapentadol prodrug or pharmaceutically acceptable salt of the present invention, and wherein upon oral administration, the prodrug or pharmaceutically acceptable salt minimizes, if not completely avoids, the gastrointestinal side effects usually seen after oral administration of the unbound tapentadol. The amount of tapentadol is preferably a therapeutically effective amount (e.g., an analgesic effective amount). The prodrug can be any tapentadol prodrug of the present invention, including compounds encompassed by Formula I.

In another embodiment of the invention, a method for increasing the oral bioavailability of tapentadol in a subject in need thereof is provided. The method comprises administering to a subject in need thereof a therapeutically effective amount (e.g., an analgesic effective amount) of a prodrug of the present invention, or a composition thereof, wherein the oral bioavailability of tapentadol provided by the prodrug is at least 10% greater than the oral bioavailability of tapentadol when tapentadol is administered alone. The prodrug can be any tapentadol prodrug of the present invention, including compounds encompassed by Formula I.

In one embodiment, a method for reducing inter- or intra-subject variability of tapentadol serum levels is provided. The method comprises administering to a subject, or group of subjects, in need thereof, a therapeutically effective amount (e.g., an analgesic effective amount) of a prodrug of the present invention, or a composition thereof. The prodrug can be any tapentadol prodrug of the present invention, including compounds encompassed by Formula I.

### Salts, Solvates and Derivatives of the Compounds of the Invention

The methods of the present invention further encompass the use of salts, solvates, of the tapentadol prodrugs described herein. In one embodiment, the invention disclosed herein is meant to encompass all pharmaceutically acceptable salts of tapentadol prodrugs (including those of the carboxyl terminus of the amino acid as well as those of the weakly basic nitrogen).

Typically, a pharmaceutically acceptable salt of a prodrug of tapentadol used in the practice of the present invention is prepared by reaction of the prodrug with a desired acid or base as appropriate. The salt may precipitate from solution and be collected by filtration or may be recovered by evaporation of the solvent. For example, an aqueous solution of an acid such as hydrochloric acid may be added to an aqueous suspension of the prodrug of a phenolic analgesic and the resulting mixture evaporated to dryness (lyophilized) to obtain the acid addition salt as a solid. Alternatively, the prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, for example, Brittain. Polymorphism in Pharmaceutical solids. Marcel Decker, New York, 1999.). The compounds useful in the practice of the present invention can have one or more chiral centers and, depending on the nature of individual substituents, they can also have geometrical isomers.

### Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier or excipient selected with regard to the intended route of administration and standard pharmaceutical practice. The compositions of the present invention also include pharmaceutically acceptable salts of the tapentadol prodrugs, as described above.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g. in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20^{th} Edition, 2000, pp. 858-914.

However, since absorption of amino acid and peptide prodrugs of tapentadol may proceed via an active transporter such as Pept1, controlled release dosage forms may be desirable, such as those which primarily release tapentadol throughout the length of the GI tract in a uniform manner. For those prodrugs of tapentadol which do not result in sustained plasma drugs levels due to continuous generation of active from a plasma reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract may also be of value.

The formulations of the present invention can be administered from one to six times daily, depending on the dosage form and dosage.

In one aspect, the present invention provides a pharmaceutical composition comprising at least one active pharmaceutical ingredient (i.e., a tapentadol prodrug), or a pharmaceutically acceptable salt thereof, and a pharmaceutically acceptable carrier or other excipient. In particular, the invention provides a pharmaceutical composition comprising a therapeutically effective amount of at least one prodrug of the present invention, or a pharmaceutically acceptable salt thereof, and a pharmaceutically acceptable carrier or excipient.

For the methods of the invention, the prodrug employed in the present invention may be used in combination with other therapies and/or active agents. Accordingly, the present invention provides, in a further aspect, a pharmaceutical composition comprising at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds must be stable and compatible with each other and the other components of the formulation. When formulated separately the compounds may be provided in any convenient formulation, conveniently in such manner as is known for such compounds in the art.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more pharmaceutically acceptable excipients or carriers. Acceptable carriers and excipients for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben).

Suitable examples of pharmaceutically acceptable stabilizers and antioxidants include, but are not limited to, ethylenediaminetetriacetic acid (EDTA), thiourea, tocopherol and butyl hydroxyan

The pharmaceutical compositions of the invention may contain from 0.01 to 99% weight per volume of the prodrugs encompassed by the present invention.

### Doses

The doses described throughout the specification refer to the amount of tapentadol in the compound, in free base form.

### Analgesia

Appropriate patients (subjects) to be treated according to the methods of the invention include any human or animal in need of such treatment. Methods for the diagnosis and clinical evaluation of pain, including the severity of the pain experienced by an animal or human are well known in the art. Thus, it is within the skill of the ordinary practitioner in the art (e.g., a medical doctor or veterinarian) to determine if a patient is in need of treatment for pain. The patient is preferably a mammal, more preferably a human, but can be any animal, including a laboratory animal in the context of a clinical trial or screening or activity experiment employing an animal model. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as, but not limited to, bovine, equine, caprine, ovine, and porcine subjects, research animals, such as mice, rats, rabbits, goats, sheep, pigs, dogs, cats, and avian species, such as chickens, turkeys and songbirds.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In a preferred embodiment, an effective amount of a prodrug of Formula I is from 5 mg to 100 mg, preferably from 5 mg to 25 mg, and more preferably from 10 mg to 20 mg. If prodrugs of Formula I provide near complete oral bioavailability, the preferred dosage is from 12.5 mg to 20 mg, based on the currently effective maximum daily doses of 50-100 mg. If the improvement in systemic availability from the prodrug yields an absolute oral bioavailability of closer to 50%, then the preferred dosage is from 25 mg to 40 mg.

Depending on the severity of pain to be treated, a suitable therapeutically effective and safe dosage, as may readily be determined within the skill of the art, and without undue experimentation, maybe administered to subjects. For oral administration to humans, the daily dosage level of the prodrug may be in single or divided doses. The duration of treatment may be determined by one of ordinary skill in the art, and should reflect the nature of the pain (e.g., a chronic versus an acute condition) and/or the rate and degree of therapeutic response to the treatment.

In the methods of treating pain, the prodrugs encompassed by the present invention may be administered in conjunction with other therapies and/or in combination with other active agents. For example, the prodrugs encompassed by the present invention may be administered to a patient in combination with other active agents used in the management of pain. An active agent to be administered in combination with the prodrugs encompassed by the present invention may include, for example, a drug selected from the group consisting of non-steroidal anti-inflammatory drugs including acetaminophen and ibuprofen or anti-emetic agents such as ondanstron, domerperidone, hyoscine or metoclopramide or unabsorbed or poorly bioavailable opioid antagonists such as naloxone or alvimopan to reduce the risk of drug abuse. In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating pain, the dose of each compound may differ from that when the compound is used alone. Appropriate doses will be readily appreciated by those skilled in the art.

### EXAMPLES

The present invention is further illustrated by reference to the following Examples. However, it should be noted that these Examples, like the embodiments described above, are illustrative and are not to be construed as restricting the enabled scope of the invention in any way.

### Example 1

### Preparation of Tapentadol Prodrugs

Step 1—Synthesis of (rac)-tapentadol hydrochloride

For the synthesis of (rac)-tapentadol hydrochloride, a route was developed starting from the commercially available ketone 3-(3-methoxyphenyl)propan-2-one. In the first step, bis(dimethylamino)methane was reacted with 3-(3-methoxyphenyl)propan-2-one, in the presence of trifluoroacetic acid, in a Mannich reaction, to give (rac)-3-(dimethylamino)-1-(3-methoxyphenyl)-2-methylpropan-1-one (Scheme 1). It was important to achieve high purity at this point since any contaminants from the starting materials could have reacted in the subsequent reaction steps. The 3-(dimethylamino)-1-(3-methoxyphenyl)-2-methylpropan-1-one (mixture of diastereoisomers) was converted to 1-(dimethylamino)-3-(3-methoxyphenyl)-2-methylpenta-3-ol using ethyl magnesium bromide in a Grignard reaction (Scheme 1). This was achieved in excellent yield without further purification.

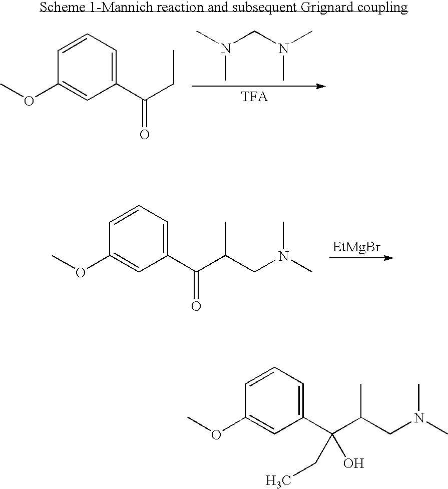

Dehydration of 1-(dimethylamino)-3-(3-methoxyphenyl)-2-methylpenta-3-ol with hydrochloric acid resulted in the formation of (rac)-1-(dimethylamino)-3-(methoxyphenyl)-2-methylpent-3-ene. Reduction of the alkene with hydrogen and catalytic palladium on carbon afforded 3-(3-methoxyphenyl)-N,N,2-trimethylpentan-1-amine as a mixture of stereoisomers (Scheme 2).

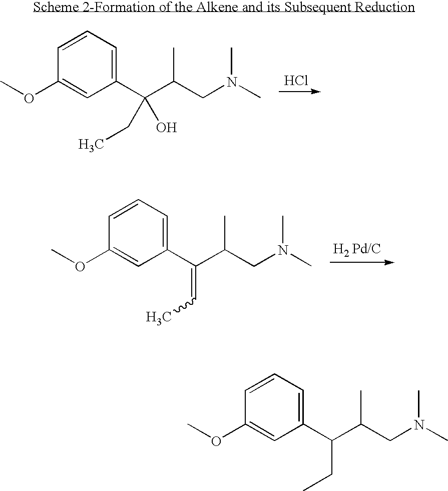

With the protected tapentadol (3-(3-methoxyphenyl)-N,N,2-trimethylpentan-1-amine) in hand, treatment with methanesulfonic acid and methionine resulted in the formation of tapentadol (Scheme 3). The free base was readily converted to its hydrochloride salt by treatment with hydrogen chloride in diethyl ether.

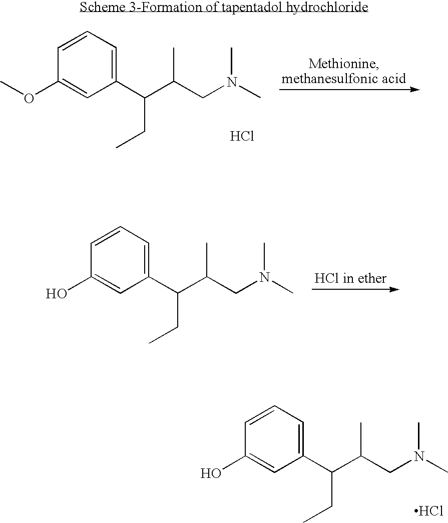

There are four stereomers of tapentadol, namely (R,R)-, (S,S)-, (S,R)- and (R,S)-isomers of which the (R,R)-isomer is currently the clinically used form.

The synthesis of tapentadol was performed without resolution of any intermediate, and it was therefore expected to yield a 1:1 (i.e., racemic) mixture of the (R,R)- and (S,S)-enantiomers. As the reduction step in the synthesis is not completely stereoselective, the (R,R)/(S,S)-mixture will be contaminated by a certain amount (<50% in total) of a 1:1 mixture of the (S,R)- and (R,S)-enantiomers which accounts for the final stereomeric ratio.

The synthesis of tapentadol described above afforded tapentadol as a mixture of diastereomers by HPLC analysis, with ca. 70% of the mixture comprising the (R,R)- and (S,S)-enantiomers. Hence, 35% of this mixture was the active (R,R)-enantiomer.

Step 2—Prodrug Synthesis using an Amino Acid (or Peptide) Tert-Butyl Ester.

Tapentadol can then be reacted with an amino acid tert-butyl ester to afford a prodrug of the present invention. This is shown below in Scheme 4, using isoleucine as an example. Briefly, (S)-isoleucine tert-butyl ester hydrochloride can be treated with diphosgene in the presence of pyridine. The resulting isocyanate can be used immediately in the next step. Reacting the isocyanate with tapentadol free-base in toluene, after column chromatography, will give the carbamate.

Subsequent deprotection with trifluoroacetic acid will yield the product as its trifluoroacetate salt.

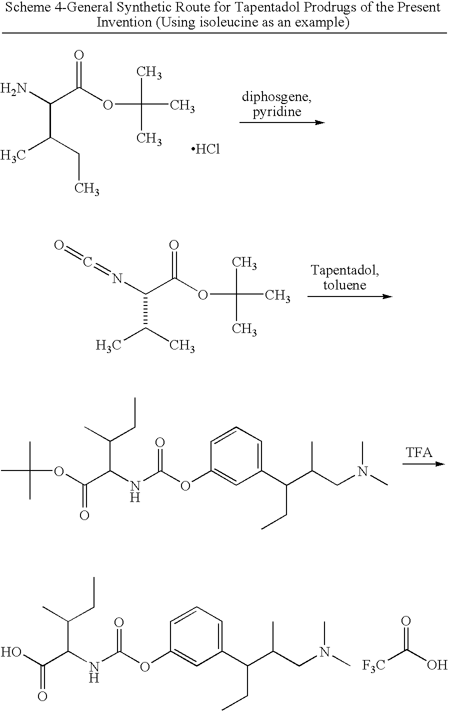

### Example 2

### Preparation of Tapentadol Valine Carbamate

The following procedure is used for the preparation of tapentadol valine carbamate. The procedure is readily amenable for the synthesis of other amino acid tapentadol conjugates, as well as tapentadol conjugates containing longer peptides.

Step 1—Synthesis of (rac)-tapentadol hydrochloride

Tapentadol hydrochloride was prepared as described in Example 1, above.

Step 2—Synthesis of (rac)-tapentadol-(S)-valine carbamate trifluoroacetate

(S)-valine tent-butyl ester hydrochloride was treated with diphosgene in the presence of pyridine and the resulting isocyanate was used immediately in the next step. Reaction with tapentadol free-base in toluene, after column chromatography, gave a modest yield of the carbamate.

Subsequent deprotection with trifluoroacetic acid yielded the product as its trifluoroacetate salt.

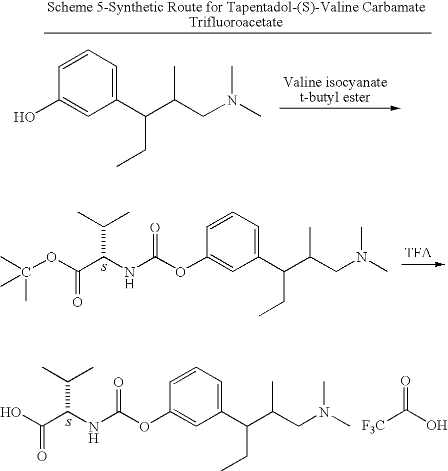

### Example 3

### Stability of Tapentadol Valine Carbamate under Conditions Prevailing in the Gut

### Methodology

Since the GI luminal stability of the tapentadol prodrugs is important if opioid-like effects on the intestinal smooth muscle are to be avoided, the rate and extent of tapentadol valine carbamate hydrolysis under the conditions prevailing in the GI tract was evaluated. If the prodrug is prematurely hydrolyzed, tapentadol would be exposed to gut opioid receptors, which could lead to a reduction in gut motility. Premature hydrolysis of the tapentadol prodrug would also negate the opportunity to deliver systemically the prodrug from which the active drug may be continuously generated.

Using USP simulated gastric and intestinal juices, the stability of tapentadol valine carbamate was investigated over a 2 hour period at 37° C. Remaining tapentadol was quantified by HPLC.

### Results

TABLE 2
Prodrug Stability in Various Media

| | Simulated gastric | Simulated intestinal | Distilled water | |
|---|---|---|---|---|
| | fluid (pH 1.1): | fluid (pH 6.8): | (pH 5.9): | pH 10.0 buffer: |
| | % remaining | % remaining | % remaining | % remaining |
| Compound | after 2 h/37° C. | after 2 h/37° C. | after 2 h/20° C. | after 2 h/20° C. |
| Tapentadol valine | 100 | 100 | 100 | 82 |
| carbamate | | | | |

As can be seen in Table 2, tapentadol valine carbamate is stable under the conditions existing in the GI tract. Thus, the compound would be expected to be absorbed intact and to have no direct effect on the opioid receptors in the gut.

### Example 4

### Comparative in vivo Bioavailability Study in the Dog

Test substances (i.e., tapentadol (1 mg/kg) and tapentadol valine carbamate (0.8 mg tapentadol base equivalents/kg)) were administered by oral gavage to a group of five dogs (dog nos. 1, 2, 3, 4 and 5) in a two-way crossover design. The characteristics of the test animals are set out in Table 3.

TABLE 3
Characteristics of experimental dogs used in study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Tables 4 and 5, below.

TABLE 4
Pharmacokinetic parameters of tapentadol following oral
administration of tapentadol HCl (1 mg tapentadol base equiv/kg)
to the dog

| Pharmacokinetic | Dog No. | | | | | | |
|---|---|---|---|---|---|---|---|
| parameter | 1 | 2 | 3 | 4 | 5 | Mean | sd |
| Cₘₐₓ (ng/mL) | 0.75 | 1.77 | 1.27 | 2.39 | 1.08 | 1.45 | 0.64 |
| Tₘₐₓ (h) | 0.5 | 0.5 | 0.5 | 1 | 0.5 | 0.5^{a} | |
| AUCₜ (ng · h/mL) | 1.84 | 1.88 | 2.68 | 4.48 | 1.92 | 2.56 | 1.13 |
| AUC (ng · h/mL) | 1.96 | 2.02 | 3.38 | 4.53 | 1.99 | 2.78 | 1.15 |
| t½ (h) | 4.0 | 0.8 | 1.7 | 1.2 | 0.8 | 1.2^{b} | |
| T_{>50%Cmax} (h) | 1.5 | 0 | 1.5 | 0.5 | 0.5 | 0.5^{a} | |

^{a}Median value
^{b}Calculated as ln2/mean k

TABLE 5
Pharmacokinetic parameters of tapentadol and tapentadol valine
carbamate (TVC) following oral administration of tapentadol
valine carbamate (0.8 mg tapentadol base equiv/kg) to the dog

| | Dog No. | | | | | | |
|---|---|---|---|---|---|---|---|
| Pharmacokinetic parameter | 1 | 2 | 3 | 4 | 5 | Mean | sd |

Tapentadol

| | | | | | | | |
|---|---|---|---|---|---|---|---|
| Cₘₐₓ (ng/mL) | 13.7 | 11.1 | 17.0 | 11.7 | 19.3 | 14.5 | 3.5 |
| Tₘₐₓ (h) | 1 | 1 | 1 | 2 | 1 | 1^{a} | |
| AUCₜ (ng · h/mL) | 29.5 | 22.8 | 36.4 | 28.3 | 43.2 | 32.0 | 7.9 |
| AUC (ng · h/mL) | 31.4 | 25.5 | 39.9 | 29.1 | 45.0 | 34.2 | 8.1 |
| t½ (h) | 0.8 | 1.1 | 0.9 | 1.0 | 0.6 | 0.8^{b} | |
| T_{>50%Cmax} (h) | 1 | 1.5 | 1.5 | 1 | 1 | 1^{a} | |
| F^{c} (%) | 2000 | 1580 | 1480 | 803 | 2830 | 1740 | 750 |

Tapentadol valine carbamate

| | | | | | | | |
|---|---|---|---|---|---|---|---|
| Cₘₐₓ (ng/mL) | 42.8 | 45.3 | 62.3 | 46.4 | 70.0 | 53.4 | 12.1 |
| Tₘₐₓ (h) | 1 | 0.5 | 1 | 2 | 1 | 1^{a} | |
| AUCₜ (ng · h/mL) | 131 | 119 | 260 | 208 | 241 | 192 | 64 |
| AUC (ng · h/mL) | 157 | 126 | 272 | 221 | 252 | 205 | 62 |
| t½ (h) | 6.7 | 3.0 | 5.0 | 5.9 | 5.3 | 4.8^{b} | |
| T_{>50%Cmax} (h) | 1.5 | 0.5 | 1.5 | 1.5 | 1.5 | 1.5^{a} | |

^{a}Median value
^{b}Calculated as ln2/mean k
^{c}Relative bioavailability was calculated after adjusting the 0.8 mg/kg of TVC to 1 mg/kg

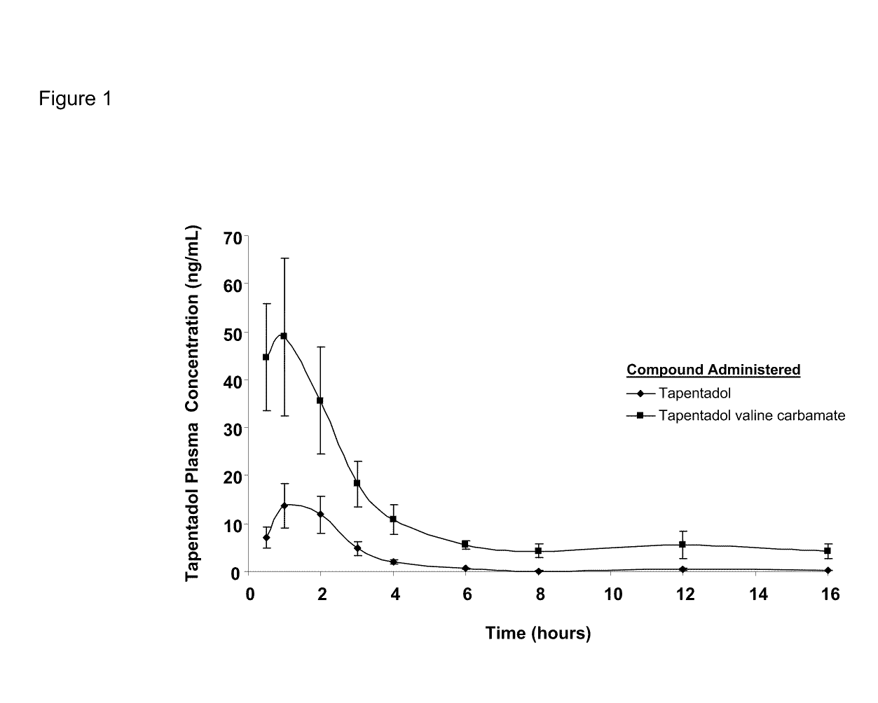

The pharmacokinetic advantages of tapentadol valine carbamate are evident in the tables above and in FIG. 1. These show a mean 17.4-fold increase in relative tapentadol bioavailability, and doubling of the duration of drug sustainment in plasma and a nearly halving of the relative standard deviation associated with Cₘₐₓ and AUC. This is expected to result in reduced inter and intra-subject variability in attained plasma drug concentrations, as well as analgesic response. A potential consequence of the less variable drug plasma levels is improved patient compliance. Additionally, the duration of sustainment of active drug in the blood was increased two to three fold following administration of the prodrug, compared to tapentadol alone, presumably as the result of continued generation of tapentadol from a plasma reservoir. Such sustainment may reduce the required frequency of administration and further aid patient compliance.

### Example 5

### Ex vivo Assessment of the Effects of Tapentadol and its Prodrug Tapentadol Valine Carbamate on Smooth Muscle Contractility in Isolated Guinea Pig Small Intestine

### Methodology

Strips of guinea pig small intestine myenteric plexus longitudinal muscle and mounted between platinum ring electrodes. The tissue was stretched to a steady tension of about 1 g and changes in force production were recorded using sensitive transducers.

The optimal voltage for stimulation was determined while the tissue was paced with electrical field stimulation (EFS) at 14 Hz, with a pulse width of 0.5 msec (Trains of pulses for 20 seconds, every 50 seconds).

EFS at optimal voltage continued throughout the protocol (stable responses=“baseline measurement of EFS”).

The 3 test conditions employed were as follows:

(1) Vehicle (deionised water, added at equivalent volume additions to test articles),
(2) Tapentadol at 6 concentrations (10 nM, 100 nM, 1 μM, 3 μM, 10 μM & 30 μM)
(3) Tapentadol valine carbamate at 6 concentrations (10 nM, 100 nM, 1 μM, 3 μM, 10 μM, & 30 μM).

Following 10 minutes of baseline EFS, first addition of test article or vehicle (deionized water) was performed.

Test concentrations were added in a non-cumulative manner with PSS washes between each addition. Next, TTX (Na+ channel blocker) was added to confirm EFS responses were elicited via nerve stimulation. EFS was then stopped.

### Results

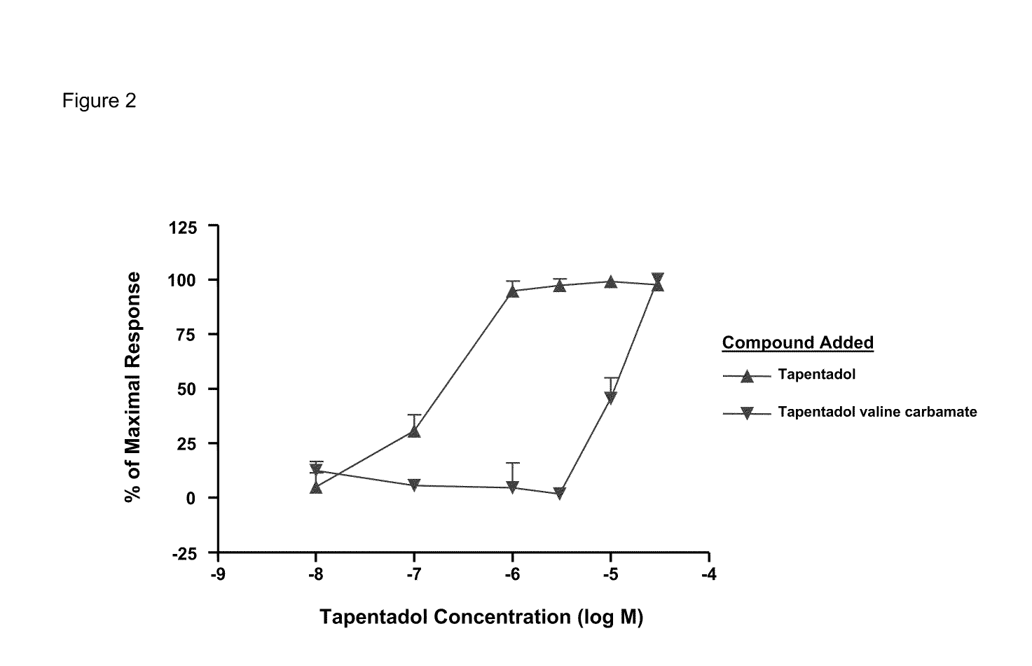

The results of these experiments, shown in FIG. 2, reveal a 50-fold reduction in the opioid effects of tapentadol valine carbamate on ileal smooth muscle compared to tapentadol itself The approximate EC₅₀ for tapentadol and the valine carbamate prodrug was 0.2 μM and 10 μM, respectively. The results indicate a potential for much less opioid mediated inhibitory effects on gut motility with the prodrug, as compared to tapentadol alone. On this basis, tapentadol valine carbamate has a much lower potential to cause constipation and other adverse GI side effects, that tapentadol itself

### Example 6

### Comparative Bioavailability of Tapentadol after Oral Administration of Tapentadol Valine Carbamate Prodrug to Rats

### Methodology

Test substances, i.e., tapentadol or tapentadol valine carbamate were administered by oral gavage to groups of male Sprague Dawley rats.

Blood samples were taken at various times after administration and submitted to analysis for the parent drug and pro-drug using a validated LC-MS-MS assay. Pharmacokinetic parameters derived from the plasma analytical data were determined using Win Nonlin.

### Results

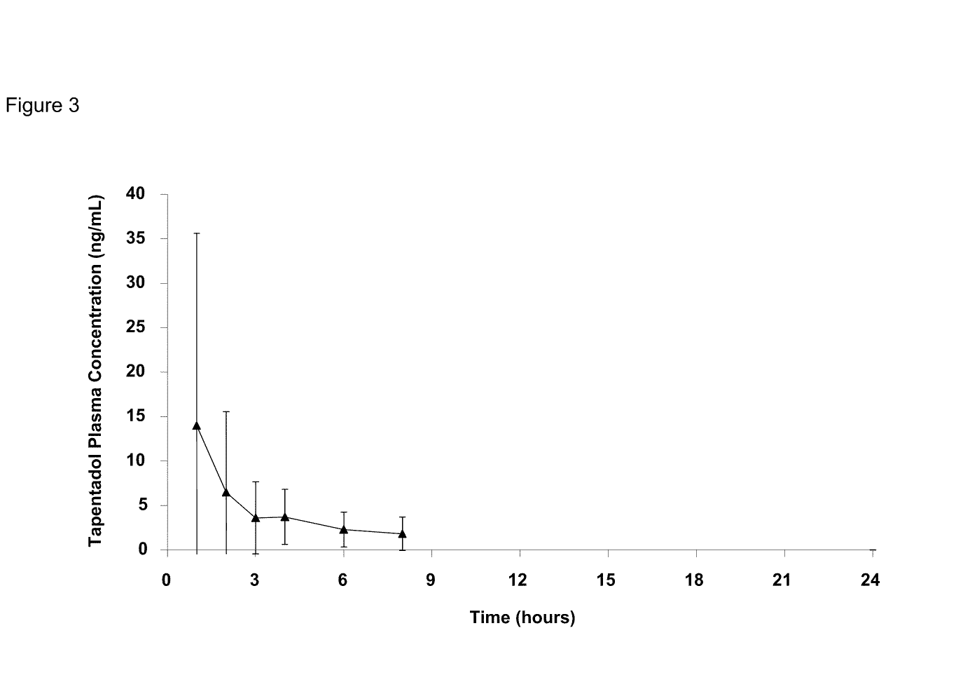

The results are given in Tables 6-9 and FIGS. 3-5.

TABLE 6
Plasma concentrations (ng/mL) of tapentadol in male rats orally
dosed with 10 mg tapentadol free base equivalents/kg

| | Animal Number | | | | | | |
|---|---|---|---|---|---|---|---|
| Time (h) | 1 | 2 | 3 | 4 | 5* | Mean | sd |
| 0.5 | 003.53 | 001.60 | 2.33 | 003.48 | 70.5 | 2.73 | 0.94 |
| 1 | 3.98 | 2.32 | 5.21 | 5.87 | 52.6 | 4.34 | 1.56 |
| 2 | 2.70 | 2.28 | 1.72 | 3.06 | 22.7 | 2.44 | 0.58 |
| 3 | 1.75 | 1.65 | 1.68 | 2.20 | 10.9 | 1.82 | 0.26 |
| 4 | 1.65 | 3.61 | 1.67 | 2.62 | 9.10 | 2.39 | 0.93 |
| 6 | 1.38 | 1.19 | 1.25 | 2.00 | 5.77 | 1.45 | 0.37 |
| 8 | 0.991 | 1.08 | 0.684 | 1.27 | 5.15 | 1.01 | 0.24 |
| 12 | BLQ | BLQ | BLQ | BLQ | 1.09 | — | — |
| 24 | BLQ | BLQ | BLQ | BLQ | BLQ | — | — |

*results not included in calculation of the mean BLQ Values less than level of quantification (LOQ, <0.5 ng/mL); excluded from calculation of the mean. Mean is not reported if results for three or more animals are below level of quantification (BLQ), or the calculated mean is BLQ

TABLE 7
Plasma concentrations (ng/mL) of tapentadol and tapentadol
valine carbamate in male rats orally dosed with tapentadol valine
carbamate at 10 mg tapentadol free base equivalents/kg

| Time | Animal Number | | | | | | |
|---|---|---|---|---|---|---|---|
| (h) | 6 | 7 | 8 | 9 | 10 | Mean | sd |

Tapentadol

| | | | | | | | |
|---|---|---|---|---|---|---|---|
| 0.5 | BLQ | 0.142 | 0.120 | 0.130 | 0.186 | 0.145 | 0.029 |
| 1 | BLQ | BLQ | 0.180 | 0.144 | 0.162 | 0.162 | 0.018 |
| 2 | BLQ | BLQ | 0.184 | 0.130 | 0.171 | 0.162 | 0.028 |
| 3 | 0.124 | BLQ | 0.163 | 0.188 | 0.140 | 0.154 | 0.028 |
| 4 | BLQ | BLQ | 0.247 | 0.283 | 0.183 | 0.238 | 0.051 |
| 6 | 0.177 | 0.169 | 3.230 | 3.730 | 1.140 | 1.689 | 1.691 |
| 8 | 0.126 | 0.162 | 1.447 | 5.110 | 1.117 | 1.592 | 2.050 |
| 12 | BLQ | 1.530 | 1.192 | 0.166 | 0.208 | 0.774 | 0.692 |
| 24 | BLQ | BLQ | BLQ | BLQ | BLQ | — | — |

Tapentadol valine carbamate

| | | | | | | | |
|---|---|---|---|---|---|---|---|
| 0.5 | 64.5 | 79.5 | 86.5 | 77.5 | 111 | 83.8 | 17.2 |
| 1 | 72.4 | 73.8 | 88.9 | 83.1 | 115 | 86.6 | 17.3 |
| 2 | 85.8 | 57.3 | 106 | 76.4 | 86.6 | 82.4 | 17.7 |
| 3 | 76.7 | 71.6 | 74.8 | 34.5 | 86.4 | 68.8 | 20.0 |
| 4 | 42.5 | 46.7 | 47.0 | 27.5 | 51.6 | 43.1 | 9.3 |
| 6 | 30.5 | 19.9 | 38.9 | 22.7 | 31.3 | 28.7 | 7.5 |
| 8 | 32.7 | 14.6 | 26.5 | 21.3 | 25.4 | 24.1 | 6.7 |
| 12 | 9.49 | 27.0 | 11.4 | 5.81 | 14.5 | 13.6 | 8.1 |
| 24 | 2.00 | 0.915 | 0.801 | 0.656 | 0.548 | 0.984 | 0.585 |

BLQ Values less than LOQ (<0.5 ng/mL); excluded from calculation of the mean.
Mean is not reported if results for three or more animals are BLQ, or the calculated mean is BLQ.

TABLE 8
Pharmacokinetics of tapentadol in male rats dosed with tapentadol
at 10 mg tapentadol free base equivalents/kg

| Pharmacokinetic | Animal number | | | | | | |
|---|---|---|---|---|---|---|---|
| parameter | 1 | 2 | 3 | 4 | 5* | Mean | sd |
| Cₘₐₓ (ng/mL) | 3.98 | 3.61 | 5.21 | 5.87 | 70.5 | 4.67 | 1.05 |
| Tₘₐₓ (h) | 1 | 4 | 1 | 1 | 0.5 | 1^{a} | |
| AUCₜ (ng · h/mL) | 15.4 | 15.3 | 14.2 | 20.6 | 151 | 16.4 | 2.9 |
| AUC (ng · h/mL) | 24.6^{c} | 18.4 | 17.4 | 27.8^{c} | 156 | 17.9 | |
| t½ (h) | 6.13^{c} | 2.30 | 3.11 | 3.83^{c} | 2.59 | 2.64 | |
| T_{>50%Cmax} (h) | 1.5 | 1.0 | NC | 1.5 | 0.5 | 1.5^{a} | |

^{a}Median value
^{b}Calculated as ln2/mean k
^{C}Extrapolated portion of AUC was >25%, therefore value is an estimate; values excluded from calculation of the mean.
*excluded from mean - considered outlier
NC Not calculable (concentrations >50% Cₘₐₓ at only one time point)

TABLE 9
Pharmacokinetics of tapentadol and tapentadol valine carbamate
in male rats orally dose with tapentadol valine carbamate at 10
mg tapentadol free base equivalents/kg

| Pharmacokinetic | Animal Number | | | | | | |
|---|---|---|---|---|---|---|---|
| parameter | 6 | 7 | 8 | 9 | 10 | Mean | sd |

Tapentadol

| | | | | | | | |
|---|---|---|---|---|---|---|---|
| Cₘₐₓ (ng/mL) | 0.177 | 1.53 | 3.23 | 5.11 | 1.14 | 2.24 | 1.95 |
| Tₘₐₓ (h) | 6 | 12 | 6 | 8 | 6 | 6^{a} | — |
| AUCₜ (ng · h/mL) | 0.604 | 3.06 | 13.2 | 12.6 | 3.40 | 9.90 | 9.32 |
| T_{>50%Cmax} (h) | NC | NC | NC | 2 | 2 | 2^{a} | — |

Tapentadol valine carbamate

| | | | | | | | |
|---|---|---|---|---|---|---|---|
| Cₘₐₓ (ng/mL) | 85.8 | 79.5 | 106 | 83.1 | 115 | 93.9 | 15.7 |
| Tₘₐₓ (h) | 2 | 0.5 | 2 | 1 | 1 | 1^{a} | 0.7 |
| AUCₜ (ng · h/mL) | 560 | 599 | 615 | 413 | 650 | 567 | 92 |
| t½ (h) | 4.08 | 3.83 | 3.16 | 3.60 | 3.06 | 3.54^{b} | — |
| T_{>50%Cmax} (h) | 2.5 | 3.5 | 3.5 | 1.5 | 2.5 | 2.5^{a} | — |

^{a}Median value
^{b}Calculated as ln2/mean k
NC Not calculable (concentrations >50% Cₘₐₓ at only one time point)

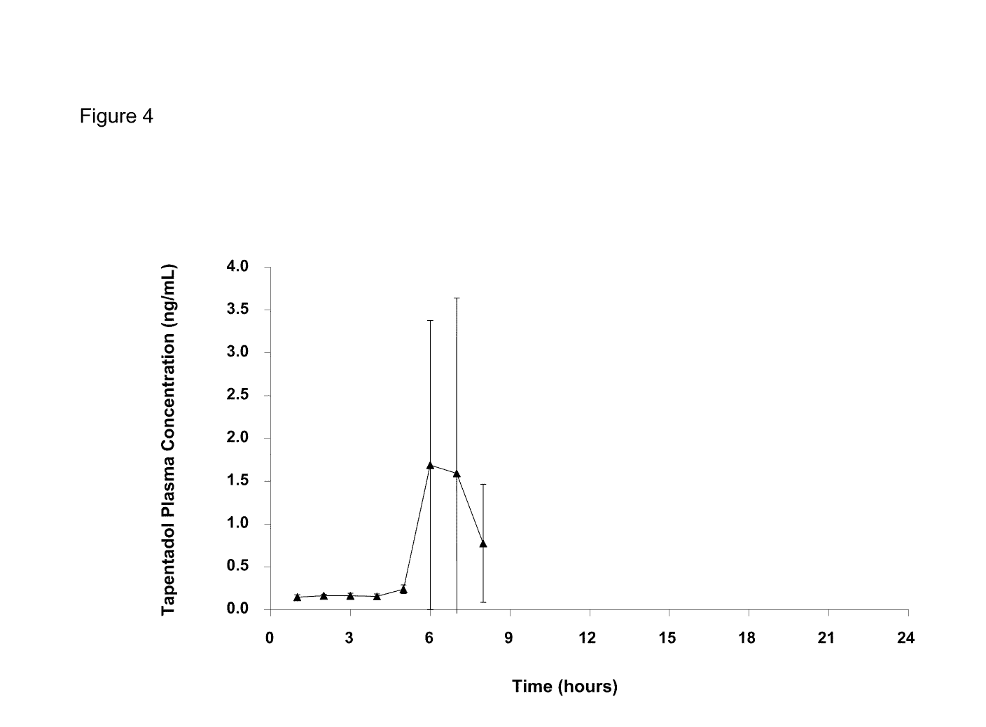

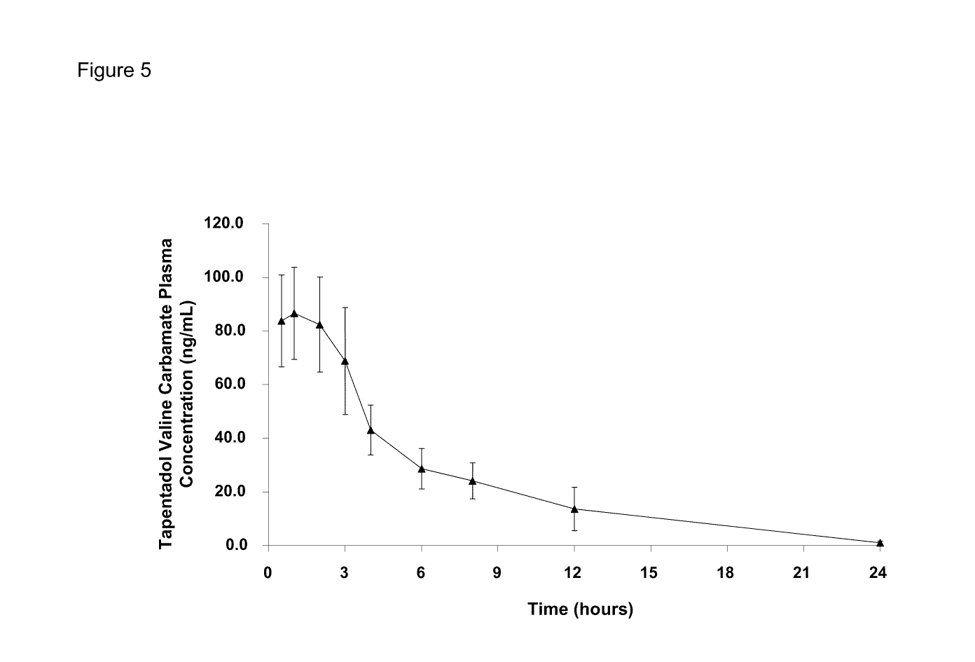

FIGS. 3 and 4 show tapentadol mean plasma concentration as a function of time after administration of either tapentadol hydrochloride (FIG. 3, see also Table 6) or tapentadol valine carbamate (FIG. 4, see also Table 7). FIG. 5 (see also Table 7) shows the mean tapentadol valine carbamate concentration in rat plasma, after oral administration of tapentadol valine carbamate (10 mg tapentadol base/kg).

After administration of tapentadol valine carbamate, high plasma levels of tapentadol valine carbamate were observed (Table 7, FIG. 5), suggesting its extensive absorption. These plasma levels persisted for several hours, potentially providing a reservoir for the continuing generation of active drug. Further, the extensive absorption of the prodrug suggests that the GI tract would not be exposed to the active drug and hence would be protected against any direct inhibitory effects on gut motility (Table 7).

Peak plasma levels of tapentadol after administration of tapentadol valine carbamate were somewhat lower than those seen after administration of the parent drug itself (Tables 8 and 9). However, there was evidence for persistence consistent with the sustainment of prodrug levels in the plasma.

### Example 7

### Comparative Bioavailability of Tapentadol after Oral Administration of Tapentadol Valine Carbamate to Monkeys

### Methodology

Test substances i.e., tapentadol and tapentadol valine carbamate were administered (either intravenously or orally) to a group of five male cynomolgus monkeys in a three way crossover design. The oral doses were given at 1 mg tapentadol base equivalents/kg while the intravenous dose was 0.5 mg/kg

Blood samples were taken at various times after administration and submitted to analysis for the parent drug and tapentadol valine carbamate using a validated LC-MS-MS assay. Pharmacokinetic parameters derived from the plasma analytical data were determined using Win Nonlin.

### Results

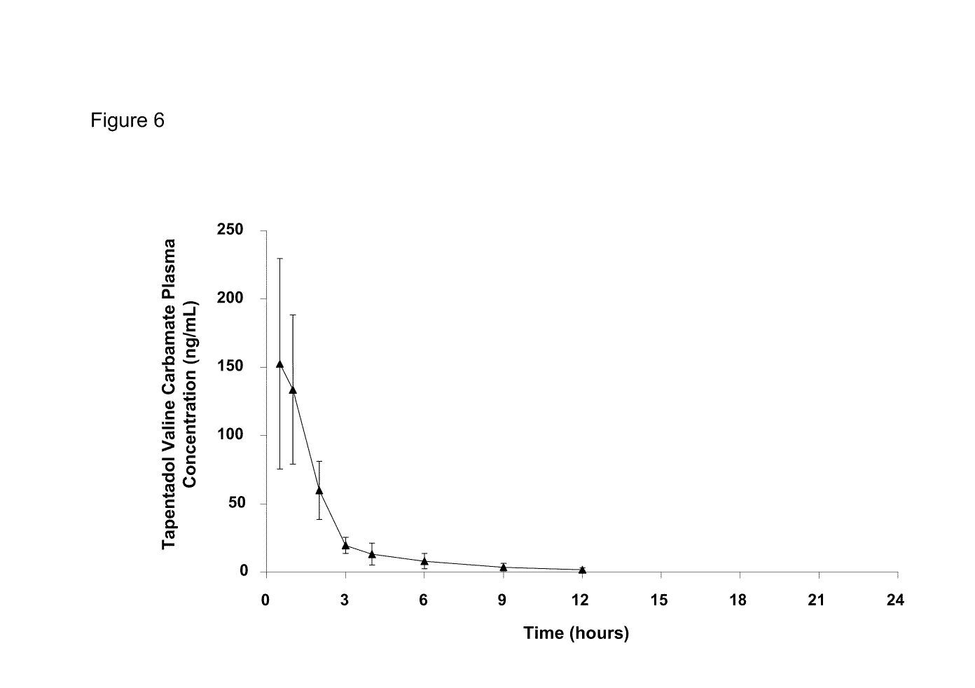

The results are given in Table 10-13 and FIG. 6.

TABLE 10
Plasma concentrations (ng/mL) of tapentadol valine carbamate
in male monkeys intravenously dosed with tapentadol valine
carbamate at 0.5 mg tapentadol free base equivalents/kg

| Time | Animal number | | | | | | |
|---|---|---|---|---|---|---|---|
| (h) | 1 | 2 | 3 | 4 | 5 | Mean | sd |
| 0 | BLQ | BLQ | BLQ | BLQ | BLQ | — | — |
| 0.083 | 790 | 951 | 914 | 1166 | 1024 | 969 | 139 |
| 0.25 | 783 | 825 | 848 | 427 | 672 | 711 | 173 |
| 0.5 | 119.8 | 576 | 620 | 609 | 430 | 471 | 211 |
| 1 | 222 | 205 | 326 | 274 | 251 | 255 | 47.6 |
| 2 | 92.3 | 112.1 | 115.5 | 85.6 | 87.7 | 98.6 | 14.1 |
| 4 | 11.8 | 20.0 | 21.4 | 12.2 | 14.6 | 16.0 | 4.5 |
| 6 | 3.21 | 4.94 | 6.10 | 3.48 | 4.20 | 4.39 | 1.17 |
| 9 | 1.04 | 0.769 | 1.89 | 1.20 | 1.35 | 1.25 | 0.419 |

BLQ Values less than LOQ (<0.5 ng/mL).
Mean is not reported if results for three or more animals are BLQ, or the calculated mean is BLQ

TABLE 11
Plasma concentrations (ng/mL) of tapentadol valine carbamate
in male monkeys orally dosed with tapentadol valine carbamate
at 10 mg tapentadol free base equivalents/kg

| Time | Animal number | | | | | | |
|---|---|---|---|---|---|---|---|
| (h) | 1 | 2 | 3 | 4 | 5 | Mean | sd |
| 0.5 | 150 | 176 | 153 | 34.7 | 249 | 153 | 77.1 |
| 1 | 96.1 | 126 | 127 | 92.1 | 227 | 134 | 54.7 |
| 2 | 31.8 | 71.3 | 58.9 | 49.4 | 87.9 | 59.9 | 21.3 |
| 3 | 10.7 | 23.1 | 25.2 | 16.4 | 22.1 | 19.5 | 5.9 |
| 4 | 5.49 | 15.1 | 25.7 | 12.7 | 6.89 | 13.2 | 8.1 |
| 6 | 4.60 | 10.0 | 17.0 | 4.51 | 3.93 | 8.01 | 5.6 |
| 9 | 1.93 | 7.34 | 5.61 | 1.11 | 1.86 | 3.57 | 2.7 |
| 12 | 0.741 | 2.97 | 3.04 | 0.716 | 0.929 | 1.68 | 1.2 |

TABLE 12
Pharmacokinetics of tapentadol valine carbamate in male monkeys
intravenously dosed with tapentadol valine carbamate at 0.5 mg
tapentadol free base equivalents/kg

| Pharmacokinetic | Animal Number | | | | | | |
|---|---|---|---|---|---|---|---|
| parameter | 1 | 2 | 3 | 4 | 5 | Mean | sd |
| C₀ (ng/mL) | 794 | 1020 | 949 | 1930 | 1260 | 1190 | 450 |
| AUCₜ (ng · h/mL) | 678 | 925 | 1040 | 912 | 843 | 880 | 130 |
| AUC (ng · h/mL | 680 | 926 | 1050 | 915 | 846 | 883 | 135 |
| t½ (h) | 1.5 | 1.1 | 1.5 | 1.5 | 1.5 | 1.4^{a} | |
| CL (L/h/kg) | 0.735 | 0.540 | 0.478 | 0.547 | 0.491 | 0.578 | 0.096 |
| Vₛₛ (L/kg) | 0.750 | 0.529 | 0.499 | 0.481 | 0.562 | 0.564 | 0.108 |

^{a}Calculated as ln2/mean k

TABLE 13
Pharmacokinetics of tapentadol valine carbamate in male monkeys
orally dosed with tapentadol valine carbamate at 1 mg tapentadol
free base equivalents/kg

| Pharmacokinetic | Animal Number | | | | | | |
|---|---|---|---|---|---|---|---|
| parameter | 1 | 2 | 3 | 4 | 5 | Mean | sd |
| Cₘₐₓ (ng/mL) | 150 | 176 | 153 | 92.1 | 249 | 164 | 57 |
| Tₘₐₓ (h) | 0.5 | 0.5 | 0.5 | 1 | 0.5 | 0.5^{a} | |
| AUCₜ (ng · h/mL) | 216 | 351 | 358 | 187 | 432 | 309 | 103 |
| AUC (ng · h/mL | 219 | 367 | 368 | 48 | 436 | 316 | 107 |
| t½ (h) | 2.28 | 3.35 | 2.48 | 1.86 | 2.88 | 2.47^{b} | |
| T_{>50%Cmax} (h) | 0.5 | 0.5 | 0.5 | 1 | 0.5 | 0.5^{a} | |

^{a}Median value
^{b}Calculated as ln2/mean k

After orally administering tapentadol valine carbamate to monkeys, substantial plasma concentrations of the tapentadol valine carbamate were achieved, which suggests that the valine carbamate prodrug was significantly absorbed (Table 11, FIG. 6). A comparison of the AUC values for the prodrug after oral dosing (Table 13) and intravenous dosing (Table 12) showed that at least 36% of the prodrug was absorbed after oral administration. The extensive absorption of the prodrug suggests that the GI tract would not be exposed to the active drug and hence would be protected against any direct inhibitory effects on gut motility.

Plasma levels of tapentadol itself after giving either the drug or the prodrug were below the quantitative limit of 0.5 ng/mL. It was therefore not possible to determine whether or not the plasma levels of tapentadol achieved after giving the prodrug were higher than those seen after giving the drug itself.

Overall however this study did provide good evidence for the efficient absorption of this prodrug in the monkey.

* * *

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of Formula I: 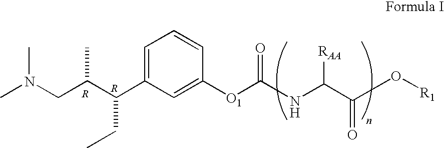
or a pharmaceutically acceptable salt thereof, wherein,
O₁ is the phenolic oxygen atom present in the unbound tapentadol;
R₁ is selected from hydrogen, an unsubstituted alkyl group and a substituted alkyl group;
n is an integer selected from 1 to 9; and
R_{AA} is a proteinogenic or non-proteinogenic amino acid side chain, and each occurrence of R_{AA} can be the same or different.

2. The compound of claim 1, wherein n is 1 and R₁ is H.

3. The compound of claim 1, wherein n is 2 and R₁ is H.

4. The compound of claim 1, wherein each occurrence of R_{AA} is a natural amino acid side chain.

5. The compound of claim 1, wherein at least one occurrence of R_{AA} is a non-natural amino acid side chain.

7. Tapentadol valine carbamate.

8. A composition comprising the compound of claim 1 and a pharmaceutically acceptable excipient.

9. A composition comprising tapentadol valine carbamate and a pharmaceutically acceptable excipient.

10. A method of treating pain with tapentadol without inducing GI side effects associated with tapentadol, comprising orally administering a tapentadol prodrug or pharmaceutically acceptable salt thereof to the subject, wherein the tapentadol prodrug is comprised of tapentadol covalently bonded through a carbamate linkage to an amino acid or peptide of 2-9 amino acids in length.

11. The method of claim 10, wherein the gastrointestinal side effect is nausea, dyspepsia, post operative ileus, vomiting, gastric ulceration, diarrhea, constipation or a combination of these side effects.

12. The method of claim 10, wherein the pain is nociceptive pain.

13. The method of claim 10, wherein the pain is neuropathic pain.

14. The method of claim 10, wherein the prodrug is tapentadol valine carbamate.

15. A method of increasing the oral bioavailability of tapentadol in a subject in need thereof, comprising administering to the subject, a tapentadol prodrug or pharmaceutically acceptable salt thereof, wherein the tapentadol prodrug comprises tapentadol bonded through a carbamate linkage to an amino acid or peptide of 2-9 amino acids in length, wherein upon oral administration of the tapentadol prodrug, the oral bioavailability of tapentadol is at least 110% the oral bioavailability of tapentadol, when tapentadol is administered in its unbound form.

16. The method of claim 15, wherein the tapentadol prodrug is tapentadol valine carbamate.
